(19) United States
(12) Patent Application Publication
(10) Pub. No.: US 2020/0016394 A1
(43) Pub. Date: Jan. 16, 2020

(54) Electrolyte Liner for Transcutaneous Stimulation Electrode

(71) Applicant: Bio-Medical Research Limited, Galway (IE)

(72) Inventor: Conor Minogue, Kinvara, Co. Galway (IE)

(21) Appl. No.: 16/335,977

(22) PCT Filed: Sep. 26, 2017
(86) PCT No.: PCT/EP17/74401

§ 371 Date: Mar. 22, 2019

(30) Foreign Application Priority Data
Sep. 26, 2016 (IE) 2016-0234

(51) Int. Cl. A61N 1/04; A61N 1/36

(52) U.S. Cl. CPC A61N 1/0496; A61N 1/36014; A61N 1/0484

(57) ABSTRACT

An electrolyte liner for use with an electrode for transcutaneous electrical stimulation, the liner comprising a flexible porous sheet of material containing a predetermined amount of liquid or gel electrolyte per unit area, said sheet being shaped to cover a skin facing surface of said electrode.

### FIELD OF THE INVENTION

The invention relates to an electrolyte liner for an electrode for transcutaneous electrical stimulation.

### BACKGROUND OF THE INVENTION

Passing an electric current through the skin involves a transduction between electron current flow in the wires and metal electrodes of the stimulator system and ionic current flow in the body. This transduction takes place partly through electrolysis and therefore an electrolyte is required at the interface between the metal (or other conductive material) electrode and the skin. Ion transport across the skin is facilitated by sweat ducts and hair follicles as well as extracellular pathways through the corneocyte matrix making up the stratum corneum. Transfer of the current into the skin also occurs by capacitive coupling across the stratum corneum, which is a thin layer having a relatively low conductivity with dielectric properties. It is usually desirable in transcutaneous stimulation that the current density be minimised since this reduces power dissipation per unit area of skin and also reduces the likelihood of stimulating pain receptors in the skin. Normally, therefore, the electrolyte needs to extend over the full area of the electrode. Moreover, to avoid hotspots, it is desirable that the current density into the skin is uniform over the contact surface area.

In the past, a water filled sponge pad was placed between the conductive surface of each electrode and the skin, with one or mores straps securing them to the body part in question. Each electrode was attached to the stimulus generator by a lead wire. Conductive gels or pastes have also been used as an electrolyte, where a film of gel is applied to the electrode before placing it on the skin and again securing it with a strap. More recently electrodes with a conductive adhesive hydrogel coating have been used, and these remain the industry standard today. They have the advantage that they adhere the electrode to the skin as well as provide the aqueous electrolyte.

Garments incorporating electrodes for transcutaneous stimulation provide a convenient way to locate a plurality of electrodes on the body with repeatable relative positioning. A garment incorporating conductive adhesive patches on the skin facing surface cannot easily slide over the skin surface, instead it must be applied in a wrapping motion such that the each electrode approaches the skin target location in a direction approximately normal to the skin surface. An example of such a garment is provided by Slendertone Flex which is an abdominal belt with three electrodes on its skin facing surface. It is applied in a wrapping motion around the abdomen.

Garments incorporating a sleeve portion through which a body part is intended to pass through are especially difficult because the inner garment surface and the skin surface have to make sliding contact during the donning and doffing process. The adhesive nature of the electrode means that it is not practical to slide an electrode into place over the skin to a target location and so a garment incorporating sleeve structures cannot easily be used.

There are parts of the body which are not suitable for a wrapping type motion. For example, a situation where electrodes have to be located at the posterior and anterior surface of each upper leg would require two wraps, one for each leg. If electrodes were required over the gluteal area, then a further wrap at that level would be required. For this anatomy, it would be far preferable to have a conventional pair of shorts that incorporate electrodes on the skin facing surface.

Hydrogel patches are also relatively expensive, and their surface becomes contaminated with skin debris. Furthermore, it is necessary to protect the adhesive surface when the device is not being used, otherwise the patch loses water through evaporation and also dust and debris can stick to the surface. Hydrogel patches are somewhat difficult to remove and replace which must be done periodically, with the attendant cost of the replacements. Furthermore, hygiene, with extended use, needs to be controlled to avoid infection. Furthermore, stimulation garments are becoming common that have multiple electrodes in close proximity to each other. It is time consuming to prepare each such electrode for use by applying a hydrogel patch, with its associated release liners and protective liners.

Various solutions have been offered to the electrode placement problem, including garments, such as shorts, with window access to the electrode sites to permit removal of protective liners from the adhesive surface of the hydrogel patch after donning. These garments are expensive and troublesome to use.

It is an object of the invention to obviate or mitigate the above drawbacks.

### SUMMARY OF THE INVENTION

According to a first aspect of the present invention, there is provided an electrolyte liner for use with an electrode for transcutaneous electrical stimulation, the liner comprising:
a flexible porous sheet of material containing a predetermined amount of liquid or gel electrolyte per unit area, said sheet being shaped to cover a skin facing surface of said electrode.

Preferably, the sheet is relatively thin.

Optionally, the sheet is characterised by a dry grammage between 40 to 100 g/m², or a thickness between 30 μm to 400 μm, or more preferably between 50 μm to 200 μm.

The electrolyte liner is preferably shaped to cover completely the skin facing surface of the electrode. In one embodiment, the electrolyte liner is shaped to extend beyond an outer periphery, preferably the entire outer periphery of the skin facing surface of the electrode.

In a preferred variation, the electrolyte liner is shaped to cover skin facing surfaces of a set of at least two or more of said electrodes. The electrolyte liner may be shaped to cover completely the skin facing surfaces of a set of at least two or more of said electrodes. In one arrangement, the electrolyte liner is shaped to extend beyond outer peripheries, preferably the entire outer peripheries, of the skin facing surfaces of a set of at least two or more of said electrodes. This allows the task of having to locate an electrolyte liner on each electrode individually to be avoided.

In an advantageous embodiment, the electrolyte liner comprises a sleeve or manifold. Preferably, the sleeve or manifold corresponds to a sleeve or manifold portion of a garment containing said electrode. In use, said sleeve or manifold is positioned between the corresponding sleeve or manifold portion of the garment containing an electrode or, preferably, at least two or more electrodes and, in use, a part of a user's body.

Preferably, the predetermined amount of liquid or gel electrolyte in the sheet per unit area is between 1 mg/cm² and 5 mg/cm², or more preferably, between 2 mg/cm² and 4 mg/cm².

Preferably, the sheet has a bulk resistivity between 0.14 Ωm to 1.2 Ωm.

In use, the electrolyte liner provides an electrolyte for conducting an electric current between the electrode and the skin.

Optionally, the electrolyte comprises preservative to inhibit microbiological growth during storage.

Optionally, the electrolyte comprises surfactants to help bind the aqueous electrolyte to the liner.

Preferably, the sheet is sufficiently soft and pliable to conform to the skin facing surface of the electrode when the sheet is placed on the skin facing surface of the electrode.

The sheet material is preferably made of textile or fabric, selected from woven or non-woven fabrics.

The sheet is preferably a disposable sheet.

Optionally, liners may be individually packed in sachets.

Optionally, liners may alternatively be provided in bulk packaging,

Optionally, the liner is stretchable.

Optionally, the liner is woven textile or fabric.

Optionally, the liner material is knitted textile or fabric. Advantageously, woven textile or fabric, especially knitted, provides particular advantages as a liner. A knitted liner is stretchable and in the form of a sleeve portion allows the liner to be pulled on over a body part which is larger than the resting dimension of the sleeve. Furthermore, the sleeve in the stretched state clings to the body part and so helps to retain it in position. It further permits fewer sizes to be made available to cover the target population because a given sleeve size can be stretched to accommodate a wider range of user sizes.

Optionally, the liner or a sleeve or manifold of a liner is provided with attachment means to keep it in place on a wearer's body, or on a garment.

Optionally, the attachment means comprises an elasticated band, for example, but not limited to a waistband for the waist or a garter for the leg or arm. Optionally, to prevent slippage of the liner on the body, silicone strips may be added to the skin-facing part of the liner. Additionally, or alternatively, the liner may be provided with a fastening mechanism to attach the liner to a garment. The one or more fastening mechanisms may comprise one or more formations or fasteners provided on the liner adapted to engage with one or more corresponding complementary formations or fasteners provided on the garment, for example but not limited to, buttons or button holes, snap fasteners, hook and loop fasteners or adhesive strips. The fabric of the liner may alternatively or in addition, be adapted to provide loop formations which engage with corresponding complementary hook formations provided on the garment.

Optionally, the textile or fabric used in the liner is adapted to retain the electrolyte solution while the liner in storage and to release it when applied to the body.

In another aspect, the invention provides a system comprising at least two or more electrodes for transcutaneous electrical stimulation and at least one electrolyte liner in accordance with the first aspect of the invention. In a preferred embodiment, the at least two or more electrodes are mounted on a skin facing surface of a garment or applicator.

In use, the electrolyte liner is placed on the skin facing surface of said electrodes.

Preferably, the electrolyte liner is shaped to cover skin facing surfaces of at least two or more of said electrodes. The electrolyte liner is preferably shaped to cover completely the skin facing surfaces of at least two or more of said electrodes. In one embodiment, the electrolyte liner is shaped to extend beyond outer peripheries, preferably the entire outer peripheries, of the skin facing surfaces of at least two or more of said electrodes.

In one arrangement, a separate electrolyte liner may be provided for each of the at least two or more of said electrodes.

In an advantageous embodiment, the electrolyte liner comprises a sleeve or manifold. Preferably, the sleeve or manifold corresponds to a sleeve or manifold portion of the garment containing said electrode. In use, said sleeve or manifold is positioned between the corresponding sleeve or manifold portion of the garment containing an electrode or preferably at least two or more electrodes and, in use, a part of a user's body.

In another aspect, the invention provides a method of estimating the area of contact of an electrode of an electrolyte liner, comprising the steps of:
applying a constant current test pulse between a pair of electrodes of the liner and measuring the resultant voltage;
measuring the rate of change of the voltage during the constant test pulse;
approximating the capacitance of the electrodes based on the measured rate of change of voltage; and
correlating the approximated capacitance with known capacitance values for area of contact data.

All essential, preferred or optional features or steps of one of the first aspect of the invention can be provided in conjunction with the features of second aspect of the invention and vice versa.

The electrolyte liner of the invention provides for greater user convenience and comfort in use, low maintenance and reduced manufacturing costs.

The invention provides an electrolyte liner and a system as defined in the appended claims.

### BRIEF DESCRIPTION OF THE DRAWINGS

Embodiments of the present invention are described hereinafter with reference to the accompanying drawings in which:

FIG. 1 shows schematically two electrodes in conductive contact with the skin through a moistened electrolyte liner sheet of the invention;

FIG. 2 shows schematically a garment incorporating a set of electrodes at fixed anatomical positions;

FIG. 3 shows schematically one of the electrodes on the skin facing surface of the garment of FIG. 2;

FIG. 4 shows schematically an electrolyte liner of the invention designed to interface between the garment shown in FIG. 2 and the body.

FIG. 5a shows schematically the electrolyte liner of the invention in place on the body, prior to donning of the garment;

FIG. 5b shows schematically the garment in place over the electrolyte liner of FIG. 5a; and

FIG. 6 shows the voltage developed across a pair of electrodes in response to a constant current pulse of 75 mA.

### DETAILED DESCRIPTION OF THE INVENTION

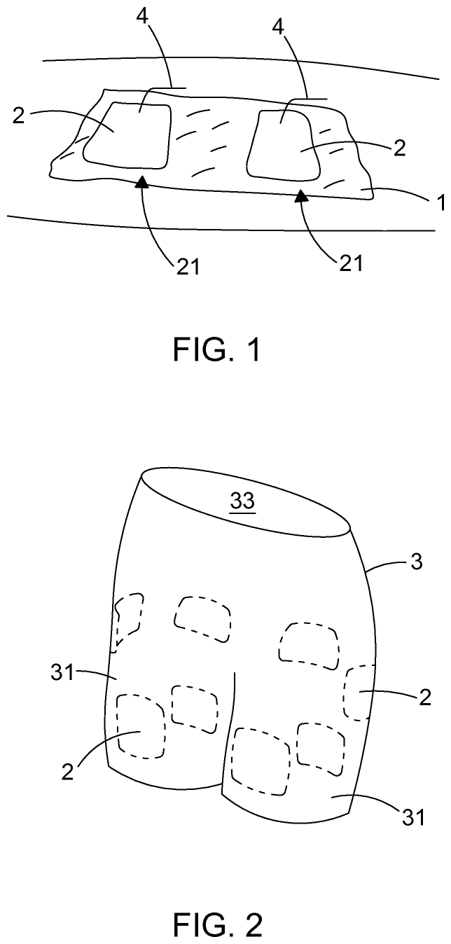

Referring to FIGS. 1 to 6, an electrolyte liner and a system in accordance with the invention will be jointly described.

The electrolyte liner 1 of the invention is provided for use with an electrode 2 for transcutaneous electrical stimulation. The electrolyte liner 1 comprises a flexible porous sheet of material containing a predetermined amount of liquid or gel electrolyte per unit area. The sheet is shaped to cover a skin facing surface 21 of the electrode 2. In use, the electrolyte liner 1 is placed on the skin facing surface 21 of the electrode 2 and provides an electrolyte for conducting an electric current between the electrode 2 and a user's skin.

The sheet is relatively thin, preferably, characterised by a dry grammage between 40 to 100 g/m², or a thickness between 30 μm to 400 μm, or more preferably between 50 μm to 200 μm.

In the presently described embodiment, as shown in FIG. 1, the electrolyte liner 1 is shaped to cover skin facing surfaces 21 of a set of at least two or more of said electrodes 2. The electrolyte liner 1 is shaped to cover completely the skin facing surfaces 21 of said electrodes 2 and extends beyond the entire outer peripheries the skin facing surfaces 21 of said electrodes 2. It will be appreciated that in other embodiments, a separate electrolyte liner may be provided for the or each electrode 2.

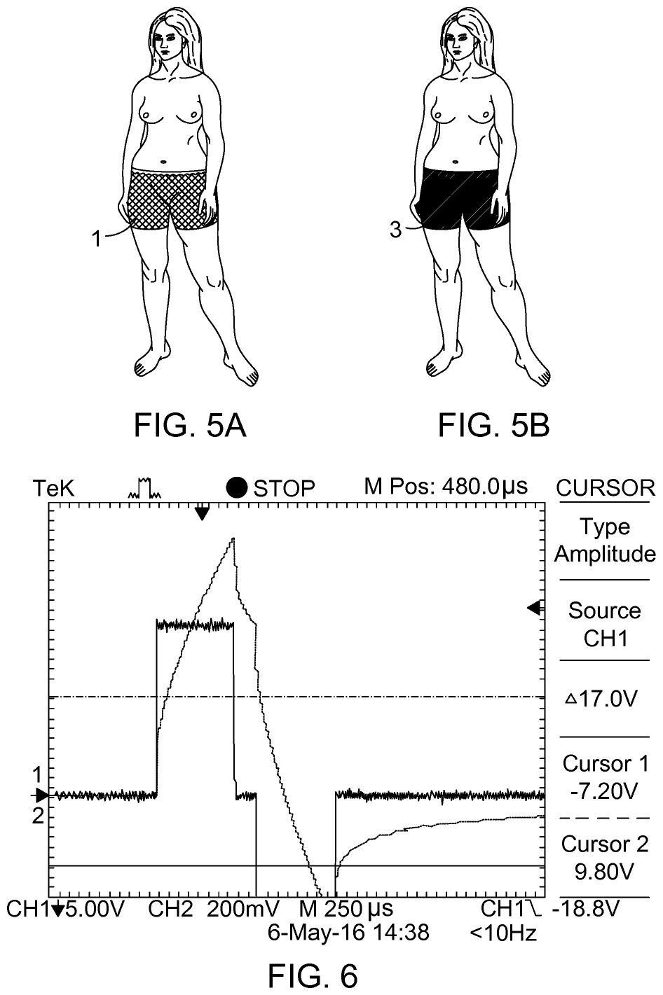

In use, the electrolyte liner 1 is overlaid onto an inner surface, i.e. a skin facing surface 33 of a garment 3 (see FIGS. 2 and 5b) as a liner, covering at least the skin facing surfaces 21 of the electrodes 2 contained in the garment 3 that are to be energised. A current is then passed into the body through the electrodes 2, the electrolyte liner 1 and the skin surface.

In a specific embodiment of the invention as shown in FIG. 2, the garment 3 contains one or more sleeve portions 31 (two are shown in FIG. 2) through which a body part passes in the course of donning and doffing. In the garment 3, electrodes 2 are located bilaterally at the anterior and posterior thigh, at the hip and overlying the lower part of the buttocks. In the presently described embodiment, as shown in FIGS. 4 and 5a, the electrolyte liner 1 comprises two sleeve or manifold portions 11 corresponding to the sleeve portions 31 of the garment 3. A main sleeve portion 15 corresponds to a lower trunk area.

The sleeve 11 of the electrolyte liner 1 can be pre-attached to the garment 3 prior to donning the garment 3. There may be occasions when it may be more convenient to apply the electrolyte liner 1 to the body part first (as shown in FIG. 5a) and then apply the garment 3 containing the electrodes 2 (as shown in FIG. 5b).

In use, said sleeve or manifold 11 is positioned between a corresponding sleeve or manifold portion 31 of the garment 3 containing the electrodes 2 and, in use, a part of a user's body. The garment 3 comprises at least two, or more, electrodes 2 mounted on a skin facing surface 33 of the garment 3.

The garment 3 is preferably designed to provide compression on body parts. When the garment 3 with the electrolyte liner 1 at the skin facing surface 3 thereof is applied to the body part, compression is provided such the each electrode 2 is pressed against the body part, compressing the moistened electrolyte liner 1. It is preferable that the garment 3 provides compression and this can be provided in a number of ways that are well known, for example in compression underwear, cycling shorts, wet suits etc. These garments use stretchable materials which are extended during the donning process and provide compression to a body part when in place. Additional compression can be provided by additional straps, tapes or strings which can be integrated into the garment 3 surrounding, or partly surrounding, the body part. A tightening mechanism can be provided which the user pulls to urge the garment 3 against the skin, especially in the area of the electrodes 2.

The predetermined amount of liquid or gel electrolyte in the sheet per unit area is preferably between 1 mg/cm² and 5 mg/cm^{2,} more preferably between 2 mg/cm² and 4 mg/cm².

Preferably, the sheet has a bulk resistivity between 0.14 Ωm and 1.2 Ωm.

The sheet is sufficiently soft and pliable to conform to the skin facing surface 21 of the electrode 2 when the sheet is placed on the skin facing surface 21.

The sheet material is preferably fabrics, selected from woven or non-woven fabrics.

The sheet is preferably a disposable sheet.

The electrodes 2 in the garment 3 take the form of conductive patches which can extend over an area from a few square centimetres up to the entire inner surface of the garment 3. More typically, for electrical stimulation the conductive patches tend to be between 25 cm² and 150 cm². The conductive patches can be made from a flexible polymer, for example silicone, loaded with conductive material such as carbon-black, silver, or carbon nanotubes. Conductive patches can also be made from conductive fabrics such as Shieldex, or by embroidery using conductive thread as demonstrated by Lawrence. Fabrics can be made conductive in selected areas by printing with stretchable conductive inks, for example Dupont PE671, applied directly onto the fabric, or onto a foundation PU layer that has been applied to the garment fabric.

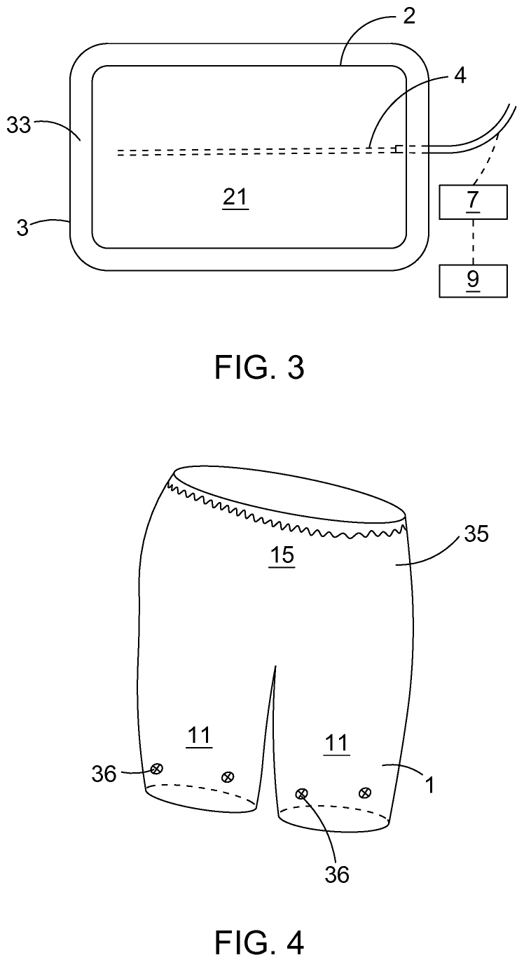

Wiring 4 embedded in the garment 3 connects each electrode 2 to a multiway connector 7 to which a stimulator module 9 is attached (see FIG. 3).

Alternatively, connections may now also be printed on fabric and encapsulated (see Dupont article “Stretchable Inks for Wearable Applications” at http://www.dupont.com/products-and-services/electronic-electrical-materials/printed-electronics/products/stetchable-inks-for-wearable-electronics.html). The wire to electrode connection can be done in various ways including crimping, over moulding, riveting or a combination of these.

It is preferable to use electrode materials which are impermeable to sweat, water and water vapour so as to retain the electrolyte between the electrode 2 and skin. Fabric electrodes can tend to wick away low viscosity electrolytes. Furthermore, the skin produces sweat continuously and this adds to the available electrolyte. It is preferable that some of this sweat is retained in liquid form between the electrode 2 and the skin.

To avoid the task of having to locate an electrolyte liner 1 on each electrode 2 individually, as described above, it is preferable that a single electrolyte liner 1 sheet covers all the electrodes 2. Naturally therefore there is tendency for current to be diverted or shunted between the electrodes 2 and not enter the body through the skin. In the present invention this is minimal provided the electrolyte liner 1 material is sufficiently thin such that the impedance into skin is very much lower that the impedance between electrodes 2 or the area of the skin contacting surface 21 of the electrode 2 is sufficient to ensure that the impedance through the tissue is much lower than the shunt path through the electrolyte liner 1. The interelectrode impedance can be increased by punching holes or slots in the electrolyte liner 1 to reduce the cross sectional area of the electrolyte liner 1 material between the electrodes 2.

The electrolyte liner 1 material is moistened with an electrolyte, that is, a fluid containing ions that act as charge carriers. Such an electrolyte is typically formed by an aqueous salt solution. It has been found that physiological saline, concentration 0.9% NaCl weight by volume has adequate resistivity, in the order of 0.14 Ωm to 1.2 Ωm. Alternatively, a salt mixture of NaCl and KCl can be used. Salt concentrations up to 5% weight by volume further boost conductivity if required. Moreover, it has been found that the moisturisation levels amounting to between 1 mg/cm² and 5 mg/cm², more preferably between 2 mg/cm² and 4 mg/cm², provide sufficient volumes of electrolyte for typical current densities used in transcutaneous stimulation using symmetric biphasic stimulation. The electrolyte is involved in an electrochemical reaction and there can be a build up of reaction by-products with associated change in pH. It is necessary to consider the resistivity of the electrolyte and the volume of electrolyte required for a given current density and duration of treatment.

It is desirable that the liner not feel wet to the user, particularly when it has to be applied in a garment. It is therefore preferable to use as little volume of electrolyte as necessary to safely complete a single session at the maximum expected current density. In one embodiment, the amount of fluid is much less than the intrinsic absorbtion capacity of the fabric. For example, 10 ml/m² to 35 ml/m² can be used in a fabric that is capable of holding 200 ml/m². An additive to adjust the viscosity of the electrolyte can further reduce the perception of wetness and moreover can retard the flow of the electrolyte within the material during storage.

The purpose of the electrolyte liner 1 material is to hold the dispersed electrolyte between the electrode 2 and the skin. The material is ideally as thin as possible but having sufficient strength not to tear or distort during donning or during use. The electrolyte liner 1 may be made of woven or non-woven textile or fabric, provided it can provide the essential requirements of retaining the required volume of liquid, whilst being sufficiently thin to have a relatively high resistance along the plane of the fabric, and sufficiently strong (e.g. material, density, weave or matrix type etc.) which meet the strength requirements to resist tearing. Non-woven material made of cotton fibre has been found to be particularly suitable because it has a high wet strength, good absorption and is biocompatible. Fibres may be treated with rewetting agents if necessary to increase hydrophilic properties. It is also well known that binders can be added to non-woven material to increase tear resistance and tensile strength.

It has been found that woven textile or fabric, especially knitted, has particular advantages as a liner. A knitted textile or fabric is stretchable and in the form of a sleeve portion 11 allows the liner to be pulled on over a body part which is larger than the resting dimension of the sleeve. Furthermore, the sleeve in the stretched state clings to the body part and so helps to retain it in position. It further permits fewer sizes to be made available to cover the target population because a given sleeve size can be stretched to accommodate a wider range of user sizes.

Preferred properties of the electrolyte liner 1 material which have been found to satisfy the above requirements are set out below:

| | |
|---|---|
| Grammage | 40 to 100 g/m² |
| Thickness | 30 μm to 400 μm, |
| | preferably 50 μm to 200 μm |
| Electrolyte content | 1 mg/cm² to 5 mg/cm², |
| | preferably 2 mg/cm² to 4 mg/cm² |
| Volume resistivity of electrolyte | 0.14 Ωm to 1.2 Ωm |
| Elongation | 10% test method ASTM D5034 |
| Grab Tensile Breaking Strength | 200 N |
| Biocompatibility | ISO 10993, skin contact 1 hr. |

It is envisaged that the electrolyte liner 1 would be used only once and then disposed. It is important that the electrolyte liner 1 contains the required amount of electrolyte and that it is evenly distributed across the material. It is envisaged that each electrolyte liner 1 would be supplied in an impermeable package such as a foil bag which is validated to prevent moisture loss over a defined shelf life period, for example 3 years. The user simply opens a pack before each session, applies the electrolyte liner 1 to the garment 3, or body part, carries out the session and then disposes of the electrolyte liner 1.

Preservative may be added to the electrolyte to inhibit microbiological growth during storage. Surfactants may be added to help bind the aqueous electrolyte to the fibre.

Optionally, the fibres of the fabric used in the liner are adapted to retain the electrolyte solution while the liner in storage and to release it when applied to the body. Release of the electrolyte is assisted by the compression applied by the garment. In the case of a stretchable knitted liner, stretching of the fabric can itself help to expel the solution from the fibres. This is especially true when the yarn of the knitted fabric is made of several fibres, or where there are cellulosic fibres having internal voids for storing electrolyte, for example cotton or viscose. Some manmade fibres have low capacity to store moisture so it is useful to use yarns that are made of a mixture of manmade and cellulosic fibres.

It is important that the electrolyte is evenly distributed in the liner when it comes to be used. Assuming it was evenly applied when manufactured it therefore requires that the electrolyte does not migrate in storage, for example by migrating under gravity to the bottom of the any packaging containing a liner, or by being squashed by having too much weight applied to said packaging. While additives can improve the binding of the electrolyte to the fabric it is nonetheless important the packaging of the liner protects against these effects. Liners may be individually packed in sachets, as is common with disposable towels. Individual packages also permit greater control of hygiene. The liners in accordance with the invention may alternatively be provided in bulk packaging, such as is common with wet wipes, however there is a risk of drying out of some units if a user leaves the package open or unsealed.

It is anticipated that occasionally the electrolyte liner 1 may become partially displaced during donning such that it does not fully cover one or more of the electrodes 2. While the user will be instructed to check each electrode 2, it is desirable that a controller (not shown) is provided to which each electrode 2 is attached to detect this fault situation, otherwise there could be an unintended increase in current density.

The impedance of each electrode 2 pair can be independently checked by passing a constant current test pulse between the pair of electrodes 2 and measuring the resultant voltage. The impedance of any one electrode 2 can be estimated by measuring the pairwise impedance of all the electrodes 2 in the array used and solving by means of simultaneous equations for each electrode 2. Furthermore the capacitance of electrode 2 to skin interface can be measured by measuring the rate of change of voltage during a constant current test pulse. The capacitance estimate gives a good indication of the area of contact of the electrode 2 and so the user can be alerted if incomplete contact if the electrode 2 is suspected. The stimulator in the invention therefore has the capacity to direct a current through any pair of electrodes 2 in the array of electrodes 2 used and to measure the resultant voltage. Techniques for this are well known in the literature, for example a bridge circuit with independent high and low side switches as described in Crowe WO2003/006106 A2.

The composition of the electrolyte, in conjunction with the electrode material, determines the electrochemical reactions involved. An aqueous solution 0.9% NaCl has been found to provide a good electrolyte in most cases. Electrolytes with higher concentrations of salt, up to 15% by weight, may penetrate into the skin and sweat ducts faster. Other salts such as KCl and CaCl₂ can be added.

FIG. 6 shows the voltage developed across a pair of electrodes 2 in response to a constant current pulse of 75 mA. The slope of the waveform allows estimation of the series capacitance of the two electrodes 2, C=I Δt/ΔV, where I is the constant current applied Δt is the time interval over which the estimate is made and ΔV is the change in voltage during that interval. This estimate is approximate as it does not take account of parallel resistance shunting the skin capacitance. The literature provides more accurate models and methods of estimating the components of the skin model.

In embodiments, a liner, or sleeve or manifold of a liner may be provided with attachment means to keep it in place on the body, or indeed on a garment. As a sleeved liner, the attachment means is may take the form of an elasticated band 35 shown by way of example in FIG. 4, such as a waistband for the waist or a garter for the leg or arm. To prevent slippage of the liner on the body, silicone strips may be added to the skin facing part of the liner as is common in hosiery.

Additionally, or alternatively, the liner may be provided with a fastening means to attach the liner to a garment. As shown by way of example in FIG. 4, the fastening means may comprise one or more formations or fasteners 36 provided on the liner adapted to engage with one or more corresponding complementary formations or fasteners provided on the garment, for example but not limited to, buttons or button holes, snap fasteners, hook and loop fasteners or adhesive strips. The fabric of the liner may alternatively or in addition, be adapted to provide loop formations which engage with corresponding complementary hook formations provided on the garment. It will be understood that attachment means and formations and fasteners may be provided at any suitable location on a liner.

In the foregoing description, unless otherwise specified the terms “electrode” and “electrodes” are used interchangeably.

Modifications are possible within the scope of the invention, the invention being defined in the appended claims.

1. An electrolyte liner for use with an electrode for transcutaneous electrical stimulation, the liner comprising:
a flexible porous sheet of material containing a predetermined amount of liquid or gel electrolyte per unit area, said sheet being shaped to cover a skin facing surface of said electrode.

2. An electrolyte liner of claim 1, wherein the sheet is relatively thin, characterised by a dry grammage between 40 to 100 g/m².

3. An electrolyte liner of claim 1, wherein the sheet is relatively thin, characterised by a thickness between 30 μm to 400 μm.

4. An electrolyte liner of claim 3, wherein the sheet is characterised by a thickness between 50 μm to 200 μm.

5. An electrolyte liner of claim 1, wherein the predetermined amount of liquid or gel electrolyte in the sheet per unit area is between 1 mg/cm² and 5 mg/cm².

6. An electrolyte liner of claim 5, wherein the predetermined amount of liquid or gel electrolyte in the sheet per unit area is between 2 mg/cm² and 4 mg/cm².

7. An electrolyte liner of any preceding claim 1, wherein the sheet has a bulk resistivity between 0.140 m and 1.20 m.

8. An electrolyte liner of claim 1, wherein the liner is made of textile or fabric, selected from woven, non-woven or knitted textiles or fabrics.

9. (canceled)

10. An electrolyte liner of claim 1, wherein the electrolyte liner comprises a sleeve or manifold.

11. An electrolyte liner of claim 10, wherein the sleeve or manifold corresponds to a sleeve or manifold portion of a garment containing said electrode, wherein said sleeve or manifold is configured to be positioned between the corresponding sleeve or manifold portion of the garment and a user's body.

12. (canceled)

13. An electrolyte liner of claim 1, wherein the electrolyte liner is shaped to cover completely the skin facing surface of the electrode.

14. An electrolyte liner of claim 13, wherein the electrolyte liner is shaped to extend beyond an outer periphery of the skin facing surface of the electrode.

15. An electrolyte liner of claim 14, wherein the electrolyte liner is shaped to extend beyond the entire outer periphery of the skin facing surface of the electrode.

16. An electrolyte liner of claim 1, wherein the electrolyte liner is shaped to cover skin facing surfaces of a set of at least two or more of said electrodes.

17. An electrolyte liner of claim 16, wherein the electrolyte liner is shaped to cover completely the skin facing surfaces of a set of at least two or more of said electrodes.

18. An electrolyte liner of claim 17, wherein the electrolyte liner is shaped to extend beyond outer peripheries of the skin facing surfaces of the set of at least two or more of said electrodes.

19. An electrolyte liner of claim 18, wherein the electrolyte liner is shaped to extend beyond the entire outer peripheries of the skin facing surfaces of the set of at least two or more of said electrodes.

20. (canceled)

21. (canceled)

22. (canceled)

23. A system comprising at least two or more electrodes for transcutaneous electrical stimulation and at least one electrolyte liner in accordance with claim 1.

24. A system of claim 23, wherein the at least two or more electrodes are mounted on a skin facing surface of a garment or applicator.

25. A system of claim 24, wherein the electrolyte liner comprises a sleeve or manifold corresponding to a sleeve or manifold portion of the garment containing said electrode and in use said sleeve or manifold is positioned between the corresponding sleeve or manifold portion of the garment.

26. (canceled)
